(19) United States
(12) Patent Application Publication
(10) Pub. No.: US 2006/0069337 A1
(43) Pub. Date: Mar. 30, 2006

(54) Patella strap and method of alleviating anterior knee pain

(76) Inventors: Bonnie Livolsi, Oceanside, CA (US); Eileen Zirpolo-Kisco, Carlsbad, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/234,445

(22) Filed: Sep. 23, 2005

Related U.S. Application Data
(60) Provisional application No. 60/613,145, filed on Sep. 24, 2004.

(51) Int. Cl. A61F 13/00; A61F 13/06

(52) U.S. Cl. 602/60; 602/61

(57) ABSTRACT

A patella strap that relieves anterior knee pain is provided. The strap includes a belt portion, and a compression member portion. The compression member includes a buttress.

### CROSS-REFERENCE TO RELATED APPLICATION

This application claims priority to provisional application Ser. No. 60/613,145, filed on Sep. 24, 2004, the entire contents of which are hereby expressly incorporated by reference.

### BACKGROUND OF THE INVENTION

1. Field of the Invention

The present invention relates to orthopedic appliances, and more particularly to orthopedic appliances for alleviating anterior knee pain.

2. Description of the Related Art

Many people suffer from anterior knee pain. A patella strap is one orthopedic device that is used to relieve anterior knee pain. The patella strap applies pressure to the patella tendon slightly superior to the tibial tuberosity.

A number of patella straps are commercially available. Examples include straps manufactured by Cho-Pat® of Hainesport, N.J., and the Kneed-It™ knee guard.

### SUMMARY OF THE INVENTION

The preferred embodiments of the present patella strap and method of alleviating anterior knee pain have several features, no single one of which is solely responsible for their desirable attributes. Without limiting the scope of this patella strap and method as expressed by the claims that follow, its more prominent features will now be discussed briefly. After considering this discussion, and particularly after reading the section entitled “Detailed Description of the Preferred Embodiments,” one will understand how the features of the preferred embodiments provide advantages, which include providing the wearer with a customized fit and uniform compression without any pressure points.

One embodiment of the present patella strap comprises a belt that is adapted to encircle a leg at a location of a patella tendon, and a compression member secured to the belt. The compression member includes a buttress that is adapted to apply compression to the patella tendon.

One embodiment of the present method of alleviating anterior knee pain comprises the step of securing a patella strap about a wearer's leg at a location of a patella tendon. The patella strap includes a belt and a compression member that are adjustable relative to one another. The method further comprises the steps of applying tension to the compression member and securing the compression member relative to the belt.

### BRIEF DESCRIPTION OF THE DRAWINGS

The preferred embodiments of the present patella strap and method of alleviating anterior knee pain, illustrating its features, will now be discussed in detail. These embodiments depict the novel and non-obvious patella strap and method shown in the accompanying drawings, which are for illustrative purposes only. These drawings include the following figures, in which like numerals indicate like parts:

FIG. 1 is a front elevational view of one embodiment of the present patella strap;

FIG. 2 is a bottom plan view of the patella strap of FIG. 1;

FIG. 3 is a rear elevational view of the patella strap of FIG. 1;

FIG. 4 is a front elevational view of the compression member of the patella strap of FIG. 1;

FIG. 5 is a top plan view of the compression member of FIG. 4;

FIG. 6 is a rear elevational view of the compression member of FIG. 4;

FIG. 7 is a front perspective view of the patella strap of FIG. 1 applied to a wearer's leg; and

FIG. 8 is a front perspective view of the patella strap of FIG. 1 applied to a wearer's leg, illustrating one step of the present method of alleviating anterior knee pain.

### DETAILED DESCRIPTION OF THE PREFERRED EMBODIMENTS

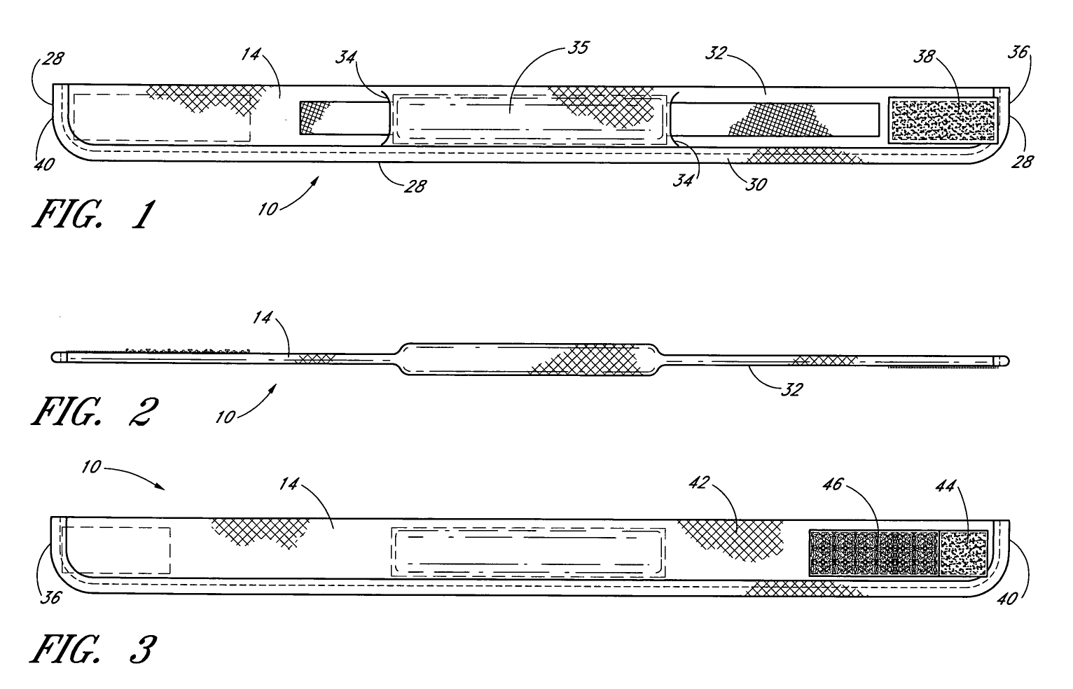

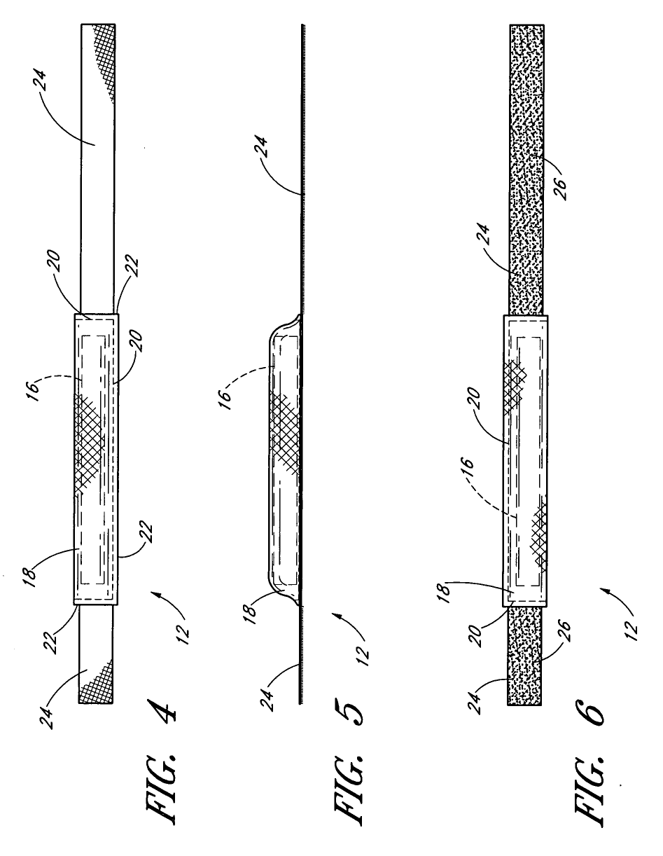

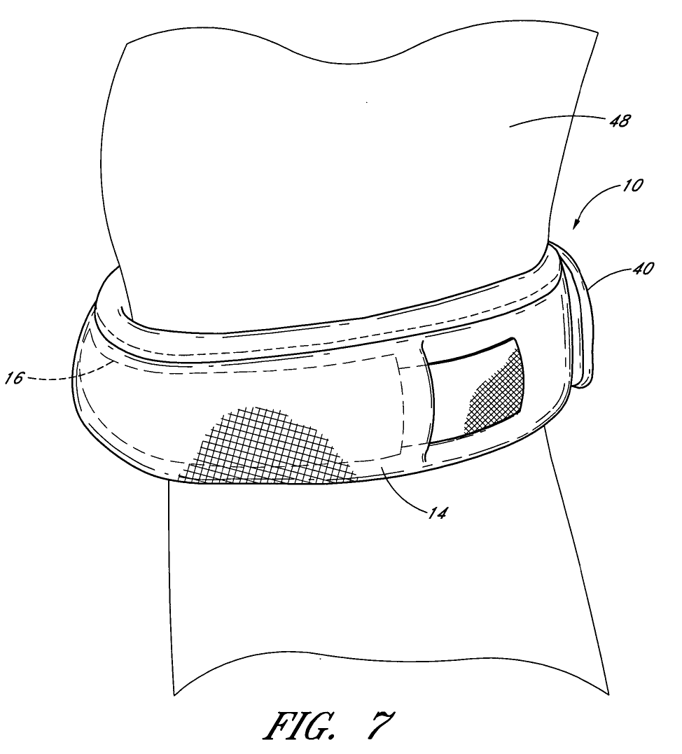

With reference to FIGS. 1-6, the present patella strap 10 includes a compression member 12 (FIGS. 4-6) that is adjustably secured within a belt 14 (FIGS. 1-3). In the illustrated embodiment, the compression member 12 comprises a buttress 16 enclosed within a pouch 18 (FIGS. 4-6). The buttress 16 may be constructed of any suitable material, such as foam, thermoplastic elastomer, neoprene, EVA, etc. The pouch 18 may similarly be constructed of any suitable material, such as a fabric. The fabric may be elastic or inelastic. Examples of suitable fabrics include nylon, LYCRA®, polyester, cotton, etc. In the illustrated embodiment, the fabric comprises a stretchable nylon. The elasticity of the pouch 18 enables the compression member 12 to provide additional compression to the wearer, as described below.

In the illustrated embodiment, the buttress 16 is substantially cylindrical. However, those of skill in the art will appreciate that the buttress 16 could be any suitable shape, such as flat or elliptical. In one embodiment, the buttress 16 comprises a flexible bladder (not shown) that contains small particles disposed within a lubricant matrix. For example, the small particles may be microspheres of glass, and the lubricant matrix may be mineral oil. Such a material has no memory. Thus, it can be deformed into virtually any shape, limited only by the shape and/or elasticity of the bladder. A buttress 16 formed of a non-shape memory material advantageously avoids the creation of pressure points on bony prominences of the wearer, thus applying uniform compression to the wearer. However, those of skill in the art will appreciate that the buttress 16 may be formed of a material that has a memory.

The pouch 18 may be a substantially rectangular section of fabric that is folded in half and closed, as by stitching 20, along the three open edges 22 (FIG. 4). Those of skill in the art will appreciate that the pouch 18 may be formed by alternative methods. In the illustrated embodiment, the compression member 12 includes first and second straps 24 secured at opposite ends of the pouch 18. The straps 24 comprise a durable material having a hook portion of a hook-and-loop fastener on a first surface 26 (FIG. 6). The straps 24 may be secured to the pouch 18 by any suitable method, such as stitching 22.

With reference to FIGS. 1-3, the belt 14 is shaped as a substantially rectangular strip. In the illustrated embodiment, the belt 14 comprises a substantially rectangular section of fabric that is folded in half and closed along three open edges 28 (FIG. 1). To protect against frayed edges, a narrow binding strip 30 overlaps the three edges 28. The binding strip 30 may be constructed of any suitable material. For example, the material may be soft and non-irritating to the wearer's skin. Examples of such a material are a 50/50 polyester/cotton blend, and polyester tricot. The outward facing surfaces of the belt 14 preferably comprise a loop portion of a hook-and-loop fastener. For example, the belt 14 may be constructed of a nylon/foam laminate.

A front surface 32 (FIGS. 1 and 2) of the belt 14 includes first and second slits 34. The slits 34 are oriented substantially perpendicular to a longitudinal axis of the belt 14. The slits 34 form ends of a pocket 35 that is adapted to receive the compression member 12. With reference to FIG. 1, the buttress 16 and pouch 18 are disposed within the pocket 35, while the straps 24 protrude from the slits 34 and overlap the front surface 32 of the belt 14.

The compression member 12 is preferably oriented such that the hook surface 26 of each strap 24 abuts the front surface 32 of the belt 14, as shown in FIG. 1. The hook surface 26 on each strap 24 is thus releasably securable to the loop surface of the belt 14. The relative positions of the compression member 12 and the belt 14 may be adjusted by first disengaging the hook surface 26 on each strap 24 from the loop surface of the belt 14, and then sliding the compression member 12 to either side with respect to the belt 14. Alternatively, the relative positions of the compression member 12 and the belt 14 may be fixed. For example, adhesive may secure the outer surface of the pouch 18 to the inner surface of the pocket 35, or stitching may secure the pouch material to the belt 14.

Near a first end 36 of the belt 14, the front surface 32 thereof includes a patch 38 of a hook portion of a hook-and-loop fastener (FIG. 1). Near a second end 40 of the belt 14, the rear surface 42 thereof includes a patch 44 of a hook portion of a hook-and-loop fastener (FIG. 3). Just inside the hook patch 44, the rear surface 42 further includes a patch 46 of a loop portion of a hook-and-loop fastener. The patches 38, 44, 46 enable the strap wearer to secure the strap 10 about his or her leg, as described in detail below.

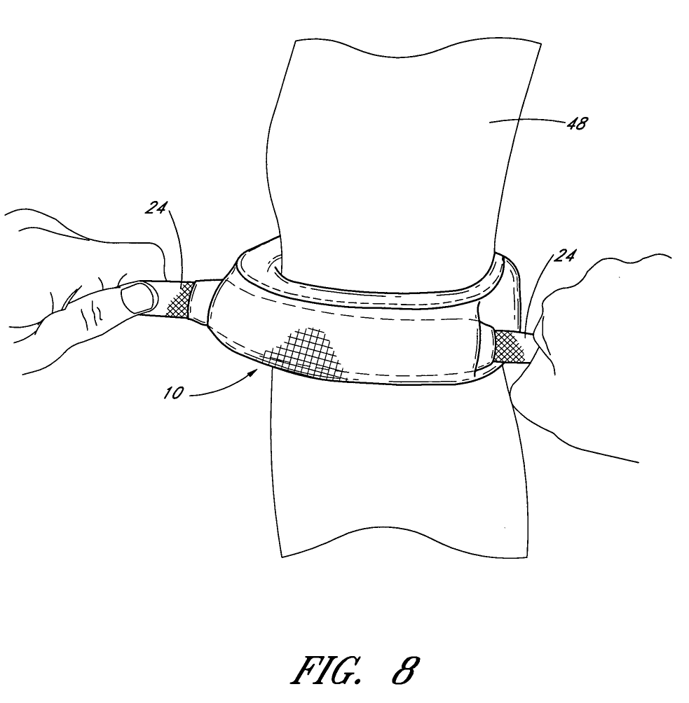

With reference to FIG. 7, to secure the present patella strap 10 about his or her leg 48, the wearer positions the belt 14 such that the buttress 16 overlies his or her patella tendon slightly superior to the tibial tuberosity. The wearer then wraps the first and second ends 36, 40 of the belt 14 around the back of his or her leg 48, positioning the second end 40 such that it overlies the first end 36. The patch 38 of hook material on the first end 36 engages the patch 46 of loop material that is secured to the second end 40 and/or the loop material of the rear surface 42 itself. The patch 44 of hook material on the second end 40 engages the loop material of the front surface 32.

The interengagement of the various portions of hook-and-loop material on the belt 14 enables the tension in the belt 14 to be adjusted in an almost limitless fashion. However, those of skill in the art will appreciate that the portions of hook-and-loop material could be replaced with alternative fasteners, such as snaps or buttons, without adversely affecting the functionality of the present patella strap 10. By wrapping the belt 14 more tightly around his or her leg 48, the wearer can apply greater pressure to his or her patella tendon. Vice versa, by wrapping the belt 14 more loosely around his or her leg 48, the wearer can apply lighter pressure to his or her patella tendon.

The relative adjustability of the compression member 12 and the belt 14 allows the wearer to adjust the position of the compression member 12 upon his or her leg 48 for proper fit and comfort. The relative adjustability also enables the wearer to apply added compression to his or her patella tendon. For example, once the present patella strap 10 has been secured to the wearer's leg 48 as described above, the wearer can then disengage the straps 24 from the front surface 32 of the belt 14, pull in opposite directions on both straps 24, and reattach the straps 24 to the front surface 32 of the belt 14. FIG. 8 illustrates this process. The pulling action creates additional tension in the pouch 18, which creates greater compression upon the wearer's patella tendon.

The dimensions of the present patella strap 10 are preferably tailored to fit a wide range of knee circumferences. For example, the strap 10 may be available in a range of sizes, such as XS, S, M, L, and XL. In one embodiment, the strap 10 is able to fit up to a 17½″ knee circumference. The buttress 16 is preferably sized to fit within the pocket of the belt 14. In one embodiment, the buttress 16 is approximately 4½″ long with a circumference of about ½″.

### Scope of the Invention

The above presents a description of the best mode contemplated for carrying out the present patella strap and method of alleviating anterior knee pain, and of the manner and process of making and using it, in such full, clear, concise, and exact terms as to enable any person skilled in the art to which it pertains to make and use this patella strap and method. This patella strap and this method are, however, susceptible to modifications and alternate constructions from that discussed above that are fully equivalent. Consequently, this patella strap and this method are not limited to the particular embodiments disclosed. On the contrary, this patella strap and this method cover all modifications and alternate constructions coming within the spirit and scope of the patella strap and the method as generally expressed by the following claims, which particularly point out and distinctly claim the subject matter of the patella strap and the method.

What is claimed is:

1. A patella strap for relieving anterior knee pain, comprising:
a belt that is adapted to encircle a leg at a location of a patella tendon; and
a compression member secured to the belt, the compression member including a buttress that is adapted to apply compression to the patella tendon.

2. The patella strap of claim 1, further comprising a pouch that encloses the buttress.

3. The patella strap of claim 2, further comprising a first strap secured to a first end of the pouch and a second strap secured to a second end of the pouch opposite the first end.

4. The patella strap of claim 3, wherein the straps are releasably securable to the belt.

5. The patella strap of claim 2, wherein the pouch is constructed of an elastic fabric.

6. The patella strap of claim 1, wherein the buttress is positioned within a pocket of the belt.

7. The patella strap of claim 1, wherein the buttress is constructed of a bladder that contains microspheres in a lubricant matrix.

8. A method of alleviating anterior knee pain, the method comprising the steps of:
securing a patella strap about a wearer's leg at a location of a patella tendon, the patella strap including a belt and a compression member that are adjustable relative to one another;
applying tension to the compression member; and
securing the compression member relative to the belt.

9. The method of claim 8, wherein the compression member includes a buttress enclosed within a pouch, and first and second straps secured to opposite ends of the pouch, and the step of applying tension to the compression member comprises pulling in opposite directions on the straps.

10. The method of claim 9, wherein the step of securing the compression member relative to the belt comprises engaging a hook fastener on the straps to a loop fastener on the belt.
